(19) United States
(12) Patent Application Publication
(10) Pub. No.: US 2008/0058915 A1
(43) Pub. Date: Mar. 6, 2008

(54) Membrane mask with acupuncture and massage functions and manufacturing methods

(76) Inventor: Chun-Min Chang, Hsinchu City (TW)

Correspondence Address: ROSENBERG, KLEIN & LEE, 3458 ELLICOTT CENTER DRIVE-SUITE 101, ELLICOTT CITY, MD, 21043, US

(21) Appl. No.: 11/515,837

(22) Filed: Sep. 6, 2006

(51) Int. Cl. A61N 1/18

(52) U.S. Cl. 607/140; 607/115; 128/907

(57) ABSTRACT

This invention is related to a facial membrane mask and its device for cosmetic and health care applications. This invention is particularly related to transcutaneous electric acupoint stimulators and cosmetic facial membrane masks. The combination of these two devices results in synergistic effects. This invention uses a facial membrane mask that can absorb skin care solution. Electrodes are integrated with the membrane at the positions which correspond to facial acupoints. When the wet facial membrane mask covers the face, the electric pulses generated by the stimulator can stimulate the corresponding acupoints, nerve or muscle. The stimulation results in acupuncture or massage effects. Therefore, both the traditional facial membrane's skin care effects, such as moisturizing and whitening etc., and acupuncture/massage effects can be obtained concurrently. Being able to modulate facial meridians and improve subcutaneous blood circulation, this invention is useful for cosmetic and health care purposes.

### BACKGROUND OF THE INVENTION

1. Technical Field

The present invention relates to a flexible membrane, which conforms to the human body's curves and, more particularly, to the flexible membrane provided with electrodes at positions corresponding to the acupoints of human body or to neuromuscular stimulation points. When the face-shaped membrane is moistened and covers the face, the electrical pulses generated by the attached pulse generator stimulate the corresponding body acupoints and neuromuscular stimulation points. This helps dredge facial meridians as well as improve blood circulation. Thus, the beneficial effects of cosmetic skin care and of electronic acupuncture/massage can be achieved at the same time. Also, this particular membrane can be designed for other human body parts whereby a user can provide skin and health care to any specific part by himself comfortably and conveniently.

2. Description of Related Articles

Various cosmetic facial masks have become increasingly popular in recent years. Especially in Taiwan, the annual consumption of such products is up to one hundred and twenty million pieces per year. Conventional mask products are typically made of absorbent materials that have been immersed in natural extracts of animals or plants or chemicals that facilitate the moisturizing, whitening and nourishing of the skin. A mask would generally have eye holes, a nose hole and a mouth hole to facilitate a user's vision, breathing and talking while in use. The desired skin care effects are achieved by applying said mask product to the face to permit sufficient contact between facial skin and the liquid contained in the mask. It is the compounds and the inorganic mineral components contained in the masks that enable such masks to give the aforementioned effects.

There are still some conventional mask products made of rubber or plastic materials. As such masks are not absorbent, instead of adhering to the face via the liquid contained in the cosmetic masks, these masks require extra belts or other means to fasten them to faces. Further, since most of such masks are formed in shell-like structure, their flexibility is very limited. It is not possible for them to conform to the user's three-dimensional face shapes and curves. Therefore, such rubber or plastic masks cannot absorb liquid and adhere closely to the facial skin. Yet another type of mask is one which uses silicon material and is equipped with an infrared light under US Patent 2004139981. However, none of the foregoing facial mask products are equipped with an meridian stimulation and massage function.

On the other hand, acupuncture and massage have a long history of use for beauty and health treatments as well as in medical and health care. However, it is not easy for the general public to practice acupuncture and massage because choosing and identifying acupuncture points which are essential to safe and efficient acupuncture and massage treatments entail experience and a lot of training.

Acupuncture can be basically divided into two types: one is invasive acupuncture which is done by applying needles to the acupoints; while the other is non-invasive acupuncture which is carried out by stimulating the acupoints on the surface of skin with electrical pulses. Invasive acupuncture may be less acceptable to the public because of hygienic concerns and psychological fears. The non-invasive acupuncture is more preferred. A non-invasive electrical transcutaneous acupoint stimulator is primarily made up of a pulse generator that provides low frequency, medium frequency or modulated electrical pulses. It also has a sticker integrated with the electrodes for attaching it to the skin. To use such a stimulator, a user must attach the electrode patch onto the skin and switch on the pulse generator to stimulate and massage his acupoints or his neuromuscular stimulation points.

Under a US2002161416 patent application, silicone rubber is used to make the upper mask, lower mask and neck mask for the forehead area, the chin area, and the neck area of the face, respectively. Metal electrodes are embedded therein for nerve and muscle stimulation purposes. An index is provided as a reference to align electrodes with stimulation points on the person's face. Because a silicone mask cannot adhere to the skin by itself, fastening gadgets such as velcro, buttons, or pins are placed in the mask structure to hold the mask in place on the wearer's face. However, since the relative positions of each electrode cannot be adjusted, it is very difficult to position the electrodes on the desired spots of the multiple acupoints when in actual use. On the other hand, since silicone rubber cannot absorb liquid, nourishing cosmetic or therapeutic materials were not and cannot be infused into such a mask. Patent numbers WO9600108 and U.S. Pat. No. 5,527,357 are related to sandwich type masks. There are a plurality of throughbores formed in an inner liner. An electrode runs through each of the throughbores. Each electrode has a leading end which is built to come in contact with the user's face. Each electrode also has an end terminal that is connected to a flexible electrical circuit board. The mask has a somewhat rigid shell and thus cannot smoothly align with the 3-D curvature of the face. In order to make the electrodes properly come in contact with the desired stimulation points, each electrode moves along its longitudinal axis so that its leading end can be positioned closely to the face of the user of the mask.

Patent US2005101994 is a facial mask with belt-like restraints at the back of the mask to fix it on the face of a wearer. It is lined with air bags. Its planar electrodes are attached to the air bags for both cheeks. The air bags are then inflated and deflated periodically to massage the face. When the air bags are inflated the electrodes are pressed against the cheeks. As a result, the electrodes can come in close contact with the skin and generate electrical stimulation effects when the electrical pulse generator is on.

Patent U.S. Pat. No. 6,694,183 is a skin cleansing device for facial skin. It includes electrode pairs attached to a facial mask and a cleansing signal generator to generate an alternating electrical cleansing signal. The electrode pairs are connected to the signal generator to apply the cleansing signal to the charged molecules of the cleansing solution located in the absorbent areas of the mask to create an alternate backward and forward motion on the facial skin surface. However, this kind of device cannot have meridian stimulation functions.

Patent JP2003339884 has a stimulator that generates alternate types of waveforms and frequencies as well as plural independent electrode stickers.

Patent U.S. Pat. No. 5,099,829 is an eye mask with a low frequency pulse generator. It has a double layer construction with a plurality of through holes, a hook and loop band for fastening the mask on the user's head and two symmetric sets of conductive projections joined together with conductive strips. A nourishing fluid cannot be applied on this kind of device. Therefore, this device cannot nourish the user's skin especially around the eye area.

In conclusion, it is obvious that there is no product yet that integrates the popular flexible membrane facial mask feature and contains cosmetic liquid with an electrical transcutaneous acupoint stimulator, to date. Current commercially available products may not yet be satisfyingly convenient or comfortable to use, not to mention its being able to provide the multiple advantages of both a membrane mask and an electrical acupoint stimulator, simultaneously.

### SUMMARY OF THE INVENTION

The present invention has been developed with the aforementioned conditions under considerations. It provides a flexible membrane which conforms to human body curves integrated with electrodes at the positions corresponding to the body's acupoints and/or neuromuscular stimulation points. As such membrane follows facial contours and is moistened as it covers the human face, the electrical pulses generated by a stimulator are applied to the corresponding body acupoints and/or neuromuscular stimulation points. These in turn help dredge facial meridians as well as improve blood circulation. Therefore, both the effects of skin care and electronic acupuncture/massage can be achieved simultaneously. Also, the enclosed membrane can be shaped for other body parts enabling the user to administer skin and health care to any of his specific body part comfortably and conveniently.

### BRIEF DESCRIPTION OF THE DRAWINGS

The novel features that are characteristic of the invention are set forth in the appended claims. The invention itself, however, as well as a preferred mode of use, further objectives and advantages thereof, will best be understood when reading the following detailed description of the invention and reading them in conjunction with the accompanying drawings below:

FIG. 1 is a plane drawing of the mask with an electrical pulse generator and electrodes in the present invention.

FIG. 2 is a schematic drawing showing the conductor wires on the mask.

FIG. 3 is a front plane drawing of the electrical pulse generator.

FIG. 4 is a rear plane drawing of the electrical pulse generator.

FIG. 5 is a plane drawing showing the mask with the ear area.

FIG. 6 is a plane drawing showing the mask with the neck area in an alternate configuration of the present invention.

FIG. 7 is a plane drawing showing the mask with the ear and neck areas in another configuration of the present invention.

FIG. 8 is a plane drawing showing the eye mask according to yet another configuration of the present invention.

FIG. 9 is a plane drawing showing the mask with an electrical pulse generator and connection to the facial membrane by electric wire.

### DETAILED DESCRIPTION OF THE PREFERRED CONFIGURATION

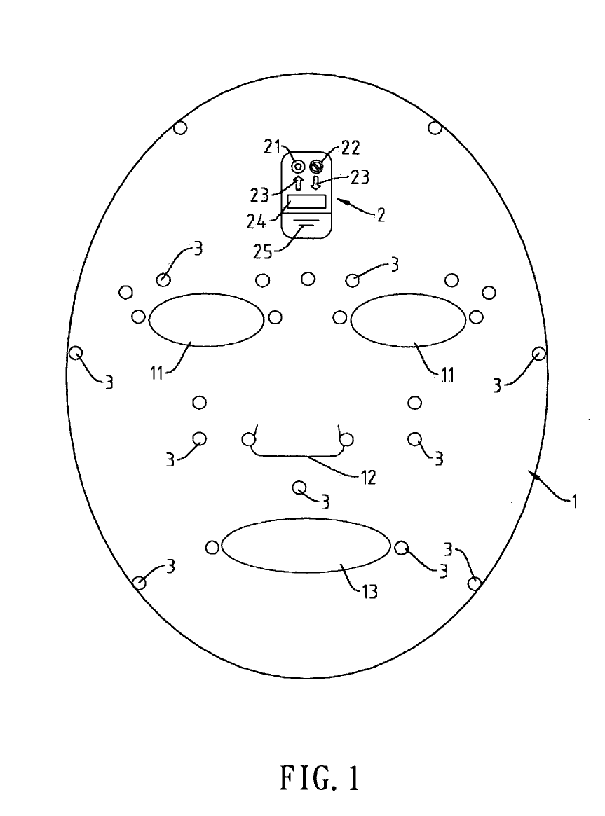

Please refer to FIGS. 1 to 9 for selected configurations of the present invention. Please take note that the figures are for purposes of illustration only and are not intended to limit the use of the invention in any way.

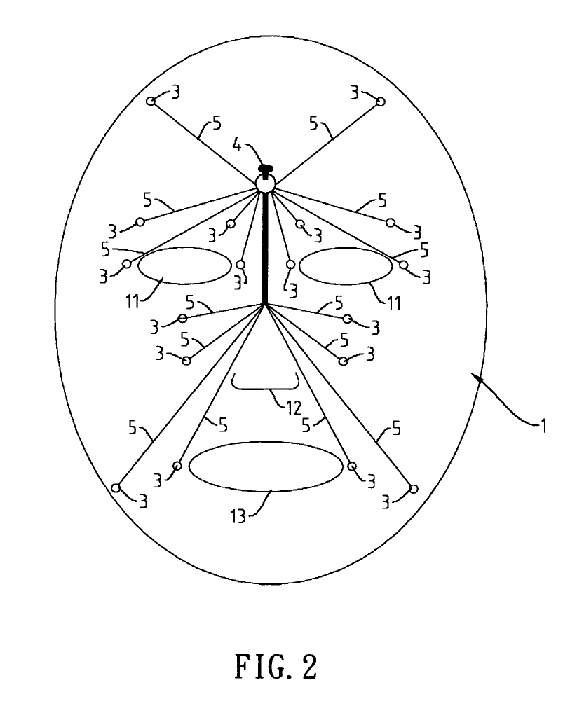

The process of manufacturing a facial membrane mask with acupuncture and massage functions, as shown in FIGS. 1 and 2, is as follows:

a mask 1, following the specific manufacturing process is flexible and capable of conforming to human body curves. The materials used can be cotton, silk, paper, flax, synthetic fiber, semi-synthetic fibers or a blend of natural and synthetic fibers. It can be non-woven, making it easy to absorb cosmetic liquids and/or other medical components; and

an electrical pulse generator 2 integrated with said mask 1 for generating the pulses that are afterwards transmitted to a network of electrodes 3 through a web of conductor wires 5. Each electrode 3 is arranged to correspond with a facial acupoint and/or neuromuscular stimulation point. Thus, the pulses generated by the electrical pulse generator 2 can be delivered to the electrodes 3 and they can in turn act on the proximal acupoints or neuromuscular stimulation points for acupuncture or massage via the mask 1. As said electrodes 3 can be positioned in relation to a pair of eye holes 11, a nose hole 12 and a mouth hole 13 on the mask 1, each electrode 3 may consequently come in contact with its corresponding acupoint when a user wears the mask 1 properly. In the event that the mask does not fit the face where it is attached to, the misalignment between the electrodes 3 and the acupoints may be adjusted easily by moving the electrodes 3 because the mask 1 is flexible. Besides, since the mask 1 can adhere closely to the skin because it is supple, the electrodes 3 integrated therein can work efficiently without auxiliary adhesives.

In FIGS. 2 and 4, a connector 4 is provided for the mask 1 to be received by a socket 26 (as shown in FIG. 4) of the electrical pulse generator 2 to conduct the electrical currents generated to the network conducting wires 5 and electrodes 3 of the mask 1. Said connector 4 has a circuit with an anode and a cathode. In the current_configuration, each conducting wire 5 that lies in between the connector 4 and each electrode 3 is insulated 5 and all the wires 5 are then gathered into a bundle and led to the central region of the mask 1 for further distribution to the points where the electrodes 3 are located.

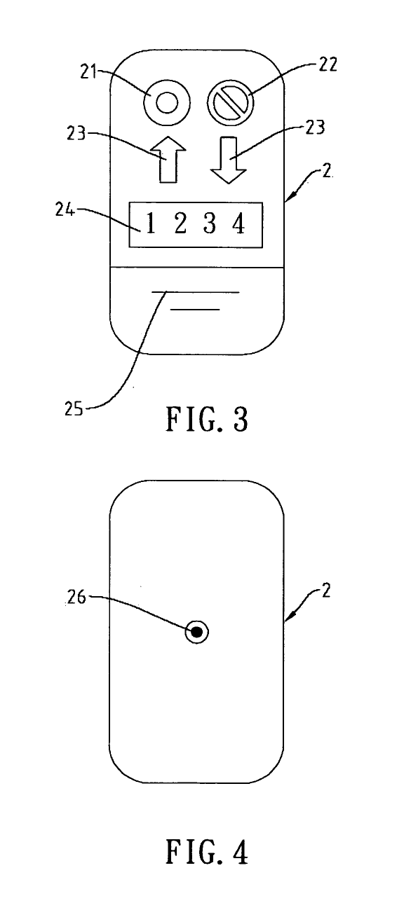

Now referring to FIG. 3, the electrical pulse generator 2 has an “ON” button 21, an “OFF” button 22, a current modulator 23 and a channel selector 24 for setting the alternative combinations of waveforms and frequencies. With the present configuration, four channels are at the electrical pulse generator 2 and each performs a set of waveforms and frequency. The electrical pulse generator 2 has a battery cover 25 which is removable for loading or unloading batteries in the battery compartment.

With the eye holes 11, nose hole 12 and mouth hole 13 provided on the mask 1, a user can properly wear the mask 1 by fitting said holes over the proper facial features to facilitate, his vision, speech and breathing. Hence, with reference to said holes, the electrodes 3 that correspond to the facial acupoints can be located. While the facial shapes and contours may differ from person to person, the locations between acupoints and facial features tend to remain the same. Because the acupoints are good conductors of electricity, the electrodes 3 in a wet mask 1 can stimulate the acupoints as long as they are placed adjacent to them. Masks 1 can be designed and manufactured in sundry sizes and shapes to meet the various needs of consumers.

Furthermore, the numerous acupoints on a human face are known to promote health, beauty and_wellness. For instance, acupoints like Zanzhu, Sizhukong, Yintang, Touwei, Yingxiang and Xiaiguan help prevent the occurrence of wrinkles and to smoothen them. Taiyang, Jiache, Yangbai, Dicang, and Quanliao help foster youthful looks. In addition, Jingming, Tongziliao, Sibai, Renzhong, Juliao, Tingggong, Baihui and Shangxing in thehead area; Fengchi, Rengying, Futu and Lianquan in the neck/head area, Yifeng behind the ears, Ermen at ragus and the eye points at earlobes, the liver and intestine points at the ear helix and the extra-ordinary acupoints on the face, head and neck areas each has its special functions. Said acupoints can be stimulated separately, jointly or alternately. Using numerous electrodes 3 allows numerous acupoints to be stimulated synchronously.

The number of electrodes 3 used on the mask 1 may come in even numbers, i.e. one or more pairs. Alternatively, electrodes 3 may also come in odd numbers if a common circuit system is employed. Each electrode 3 is connected with the electrical pulse generator 2 by way of some conductive material at one end while the rest of the parts are in contact with the skin in a direct way or in an indirect way through the mask 1 which is located in-between. Further, each electrode 3 may be made of a conductive material such as metal, metallic oxide, carbon powder,conductive rubber or other conductive polymers. However, the preferred metal conductor is silver, copper and an alloy thereof; the preferred metallic oxide is zinc oxide and zirconium oxide; the preferred rubber is karaya gum and other silicones; while the preferred polymer is poly(acetylene), poly(pyrrole), poly(thiophene), poly(aniline),and poly(para-phenylene vinylene). A conductive material made from a combination of the above mentioned materials may also be used.

The electrode 3 may also be a printed circuit made from a conductive material. In other words, each electrode 3 in the form of a printed circuit may be directly printed on the mask 1. The acceptable materials of said printed electrodes 3 include a conductive ink, such as silver, copper, conductive zinc oxide, conductive zirconium oxide, conductive carbon powders, conductive polymers, conductive rubber, conductive silicone, and a combination of the foregoing materials.

The way of fastening each electrode 3 with the mask 1 may be done by sewing, pasting or interlaying. Another alternative is to form pockets on the mask 1 and then to place the electrodes 3 inside. In case of a printed electrode, this can be simply printed on the mask 1.

Each wire conductor 5 which is connected to each electrode 3 and with the electrical pulse generator 2 can be a flexible insulated wire, made of metal, metallic oxide, carbon powder, rubber or polymers. Each wire conductor 5 may be attached to the mask 1 by sewing, pasting or interlaying. In the case of a printed circuit 5, it can be directly printed or electroplated on the mask 1. Acceptable materials for said printed electrodes 3 or circuits 5 include a conductive ink, such as silver, copper, conductive zinc oxide, conductive zirconium oxide, conductive carbon powders, conductive polymers, conductive rubber, conductive silicone, and a combination of the foregoing materials.

Whereas the preferred metals are silver ink or copper ink. However, silver ink is the most preferred.

As to the printing methods, screen printing and ink jet printing can be used for automatic production. An insulating strip may be printed with the selected conductor material first and then laminated with the mask 1. Or, in an alternative method regarding multi-layer printing, the mask 1 is first printed an insulating strip, then printed with the conductor circuit. The conductor circuit is further covered by another insulating material by printing. Said insulating layers are characterized by being flexible non-conductors. It is best that the insulating layers are also characterized by being an anti-oxidant. As such they can help protect the conductor circuit from becoming oxidized or corroded. Each set of electrodes 3 and wire conductors 5 can be integrated with or printed on the mask 1 simultaneously. The electrodes 3 and wire conductors 5 may possibly come as separate parts and be assembled by welding or through a connector. The connection between wire conductors 5 on the mask and the electrical pulse generator 2 may be a button-like, magnetic, or a pin-type connector, or other electronic connectors. In the case of a printed circuit system, it is appropriate to have a printed circuit connector. Furthermore, one can use a connector with series ports for multiple channels. Each channel performs a set of waveforms and frequency.

The electrical pulse generator 2 may be a low frequency transcutaneous nerve stimulator, a modulated middle-frequency electrical stimulator or a vector transcutaneous stimulator of any type, or for convenience, a mini transcutaneous stimulator. The pulses generated by the electrical pulse generator 2 may be an effective pulse type or mixed pulse types, where a low frequency between 1˜200 Hz is preferred ; a modulated middle-frequency between 1 KHz˜10 KHz is used for deeper penetration. The pulse width usually is between 10˜1000 microseconds. The pulse strength should depend on its acceptability by the body. Various pulse combinations may be provided to reduce the users' tolerance and therefore enhance the treatment efficiency.

Traditional batteries, button batteries, flexible plastic batteries or polymer lithium batteries can power the electrical pulse generator 2.

The mask shape should not be limited to the appearance shown in FIGS. 1 and 2. The mask of the present invention may have holes at the positions corresponding to a person's eyes, nose and mouth. The mask may also cover the face entirely or only partially or even be extended to cover the neck or ear areas.

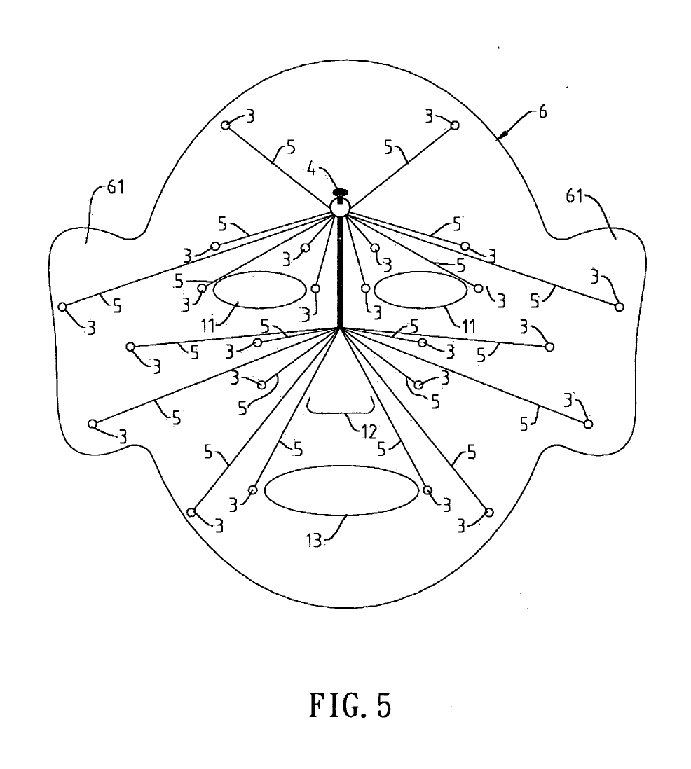

It is obvious that there shall be more variations to the present invention that can be developed. FIG. 5 is provided to illustrate a second configuration of the present invention. Here, the mask 6 is extended to cover the ear area 61 and thus relevant acupoints on the ears such as Yifeng behind the ear area, Ermen at tragus, eye points at the earlobes, as well as the liver and intestines points at the ear helix can be covered by the mask 6.

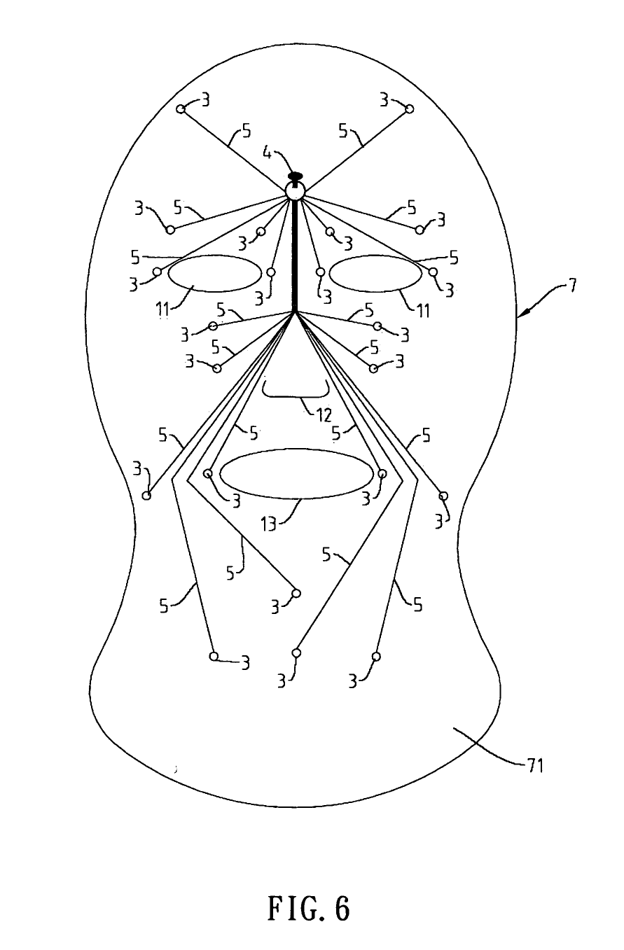

A third configuration of the present invention is further explained through FIG. 6. It is apparent that the mask 7 comprises a neck portion 71 to cover the relevant acupoints on the neck area such as Fengchi, Rengying, Futu, and Lianquan.

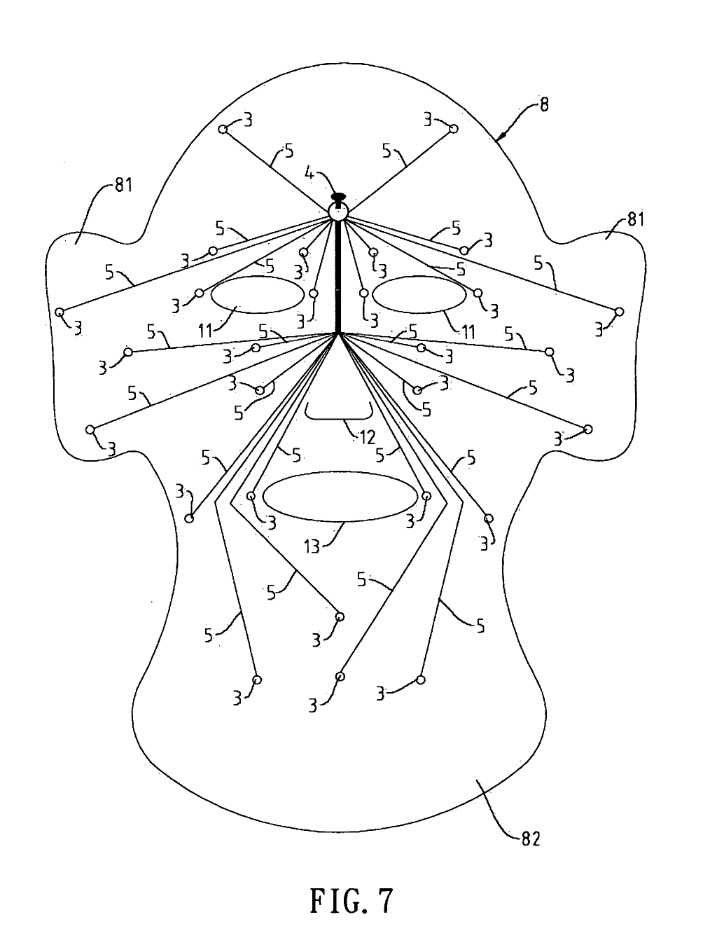

In FIG. 7, what is illustrated is a fourth configuration of the present invention. The disclosed mask 8 includes ear parts 81 and neck parts 82 to cover the ears and neck as well as other related acupoints.

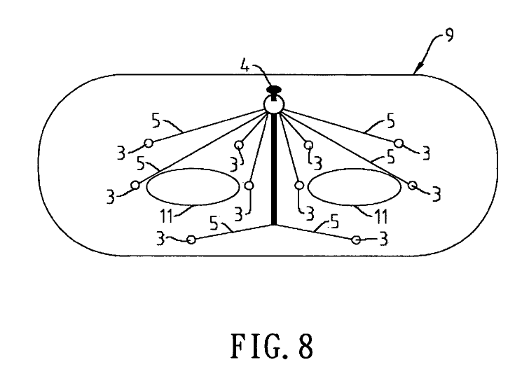

Reference is now made to FIG. 8 for a fifth configuration of the present invention. Through such a mask, 9 that is specially designed for the eye area, it is to be understood that the disclosed subject matter can be designed to cover only a specific segment of a person's face.

To sum up the above description, despite the shape of the mask, the electrodes 3 thereof can be arranged to correspond to the acupoints of the area which the mask covers. The disclosed subject matter can also be shaped to conform to other human body areas and installed with electrodes 3 at the positions corresponding to the relevant acupoints. For example, when the disclosed subject matter is utilized to treat the breasts, it may massage and in turn plump up the breasts. As the disclosed subject matter is utilized to treat the shoulders or waist, it can ease muscle aches. In cases when it is used to treat the knees or other joints, it can help prevent or alleviate osteoarthritis and relax aching joints. Extra skin care materials or medical preparations can be added to the contraption depending on the applied object. For instance, anodyne may be infused to treat arthralgia in together with the effects of acupuncture/massage and transcutaneous medication.

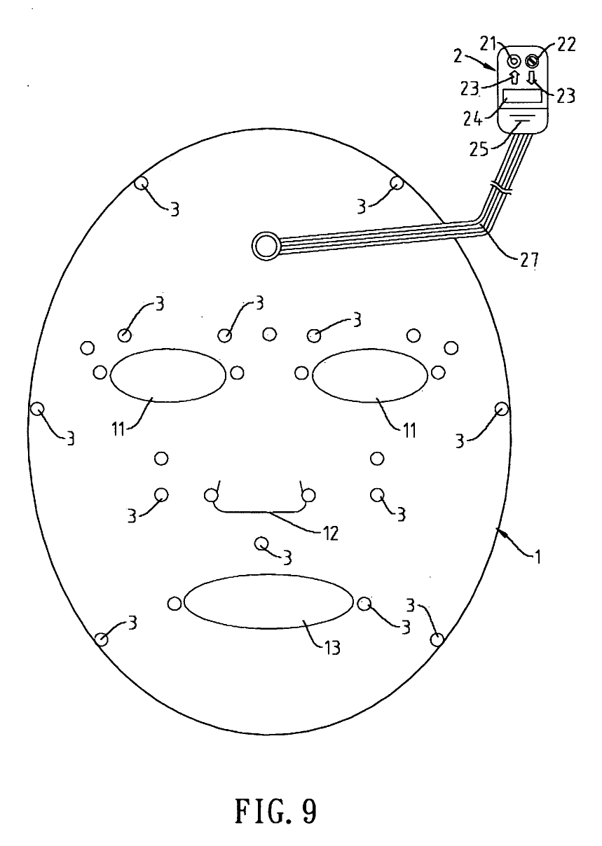

FIG. 9 further illustrates a sixth configuration of the present invention. Here, the mask 1 is connected to the electrical pulse generator 2 via an electrical wire.

Although special versions of the invention have been described in detail for illustrative purposes, it must be understood that other versions may be made possible with the scope of the invention herein described.

What the invention claims is as follows:

1. A facial membrane mask with acupuncture and massage functions comprising:
a human face-shaped membrane which is flexible and conforms to facial curves and is capable of adhering to the face;
an electrode on the membrane capable of receiving an electrical current and afterward stimulating an acupoint or a neuromuscular stimulation point that can deliver the effects of acupuncture and massage.

2. The mask as stated in claim 1, which makes use of materials selected from the group consisting of cotton, silk, paper, flax, synthetic fiber, semi-synthetic fiber, and a blend of natural and synthetic fibers.

3. As in claim 1, the connector for receiving the electrical pulse current is a button-like connector, a magnetic connector, a pin connector, or a printed circuit connector.

4. As in claim 1, among the electrodes and connectors, a wire conductor is made from a metal conductor, a conductive rubber or a conductive polymer.

5. As in claim 1, the electrodes and wire conductors are constructed by a printed circuit.

6. As in claim 5, the printed material of electrodes and wire conductors is selected from the group consisting of silver ink, copper ink, conductive zinc oxide, conductive zirconium oxide, conductive carbon powders, conductive polymers, conductive rubber, conductive silicone, and a combination of the foregoing materials.

7. As in claim 1, the membrane mask can cover the human face either entirely or partially.

8. As in claim 7, the membrane mask is designed for human eye area treatment.

9. As in claim 1, the membrane mask can cover the face and ear areas.

10. As in claim 1, the membrane mask can cover the face, neck and ear areas.

11. A membrane with acupuncture and massage functions, comprising:
a membrane which is flexible and adaptable to body curves and capable of adhering to the skin surface;
an electrode on the membrane to receive an electrical pulse and afterwards stimulate an acupoint or a neuromuscular stimulation point to achieve the effects of acupuncture and massage; and
where the shape of the membrane conforms to a body area.

12. The membrane with acupuncture and massage functions as in claim 11, where the membrane contains skin care liquids or medical preparations.

13. The membrane with acupuncture and massage functions as in claim 11, where the electrodes and wire conductors are constructed by a printed circuit.

14. A device with acupuncture and massage functions comprising:
a cover membrane which is flexible and adaptable to body curves and capable of adhering to skin surface;
an electrode on the membrane to receive an electrical pulse current and afterwards stimulate an acupoint or a neuromuscular stimulation point to achieve the effects of acupuncture and massage;
an electrical pulse generator, which is electrically connected to the network of electrodes through a web of conductors and transmits an electrical pulse current generated thereby to the web of electrodes through the conductors; and
where the shape of the membrane conforms to a body area.

15. As in claim 14, wherein the electrical pulse generator is selected from the group consisting of a low frequency transcutaneous stimulator, a modulated middle-frequency electrical stimulator, a vector transcutaneous stimulator and a mini transcutaneous stimulator.

16. As in claim 14, the pulses generated by the electrical pulse generator are a combination of at least one waveform and frequency.

17. As in claim 14, the membrane is connected with the electrical pulse generator through an electric wire.

18. A method of manufacturing the membrane with acupuncture and massage functions, comprising:
installing at least one electrode on a membrane which is flexible and adaptable to body curves and capable of adhering to the skin surface;
shaping the membrane to conform to a body area; and
where the electrode is capable of receiving an electrical pulse and afterwards stimulate an acupoint or a neuromuscular stimulation point to achieve the effects of acupuncture and massage.

19. The method of manufacturing a membrane with acupuncture and massage functions as in claim 18, at least one wire conductor is installed thereon.

20. The method of manufacturing a membrane with acupuncture and massage functions as in claim 19, the electrode and wire conductor are installed by means of screen printing or ink jet printing.
